(19) United States
(12) Patent Application Publication
(10) Pub. No.: US 2025/0160402 A1
(43) Pub. Date: May 22, 2025

(54) AEROSOL PROVISION DEVICE

(71) Applicant: NICOVENTURES TRADING LIMITED, London (GB)

(72) Inventors: Mark POTTER, London (GB); Richard HAINES, London (GB); Gareth JAMES, London (GB)

(21) Appl. No.: 18/840,993

(22) PCT Filed: Feb. 24, 2023
(86) PCT No.: PCT/EP2023/054727

§ 371 Date: Aug. 23, 2024

(30) Foreign Application Priority Data
Feb. 25, 2022 (GB) 2202623.1

(51) Int. Cl. A24F 40/40; A24F 40/50

(52) U.S. Cl. CPC A24F 40/40; A24F 40/50

(57) ABSTRACT

An aerosol provision device including an aerosol chamber moveable between a first position wherein the aerosol chamber is in contact with an aerosol generating article and a second position wherein the aerosol chamber is not in contact with the aerosol generating article. The aerosol provision device further includes a first mechanism arranged to move the aerosol chamber between the first and second positions.

### RELATED APPLICATIONS

The present application is a National Phase entry of PCT Application No. PCT/EP2023/054727 filed Feb. 24, 2023, which claims priority to GB Application No. 2202623.1 filed Feb. 25, 2022, each of which is hereby incorporated by reference in their entirety.

### TECHNICAL FIELD

The present invention relates to an aerosol provision device, an aerosol generating system and a method of generating an aerosol.

### BACKGROUND

Smoking articles such as cigarettes, cigars and the like burn tobacco during use to create tobacco smoke. Attempts have been made to provide alternatives to these articles by creating products that release compounds without combusting. Examples of such products are so-called “heat not burn” products or tobacco heating devices or products, which release compounds by heating, but not burning, material. The material may be, for example, tobacco or other non-tobacco products, which may or may not contain nicotine.

Aerosol provision systems, which cover the aforementioned devices or products, are known. Common systems use heaters to create an aerosol from a suitable medium which is then inhaled by a user. Often the medium used needs to be replaced or changed to provide a different aerosol for inhalation. It is known to use induction heating systems as heaters to create an aerosol from a suitable medium. An induction heating system generally consists of a magnetic field generating device for generating a varying magnetic field, and a susceptor or heating material which is heatable by penetration with the varying magnetic field to heat the suitable medium.

Conventional aerosol provision devices comprise a cylindrical heating chamber into which a rod shaped consumable is inserted.

Next generation devices are contemplated wherein a consumable having a shape other than cylindrical is used, such as a consumable comprising a planar substrate. The planar substrate may comprise a susceptor to be heated by penetration with a varying magnetic field. For example, the planar substrate may comprise a card base layer having an aluminium foil layer adhered thereto. The aluminium foil layer may act as a susceptor. An aerosol generating material (i.e. gel) may be provided upon the aluminium foil layer (susceptor). The planar substrate may be inserted into an aerosol provision device and may be translated or rotated relative to a heating element.

### SUMMARY

According to an aspect there is provided an aerosol provision device comprising:
an aerosol chamber moveable between a first position wherein the aerosol chamber is in contact with an aerosol generating article and a second position wherein the aerosol chamber is not in contact with an aerosol generating article; and
a first mechanism arranged to move the aerosol chamber between the first and second positions.

According to various embodiments an aerosol provision device is provided wherein an aerosol chamber may be moved into contact with an aerosol generating article when it is desired to generate a puff of aerosol from the aerosol generating article. The aerosol chamber may then be disengaged from the aerosol generating article thereby permitting rotation or translation of the aerosol generating article. As a result, a fresh portion of aerosol generating material may then be moved into proximity of an inductive heating element enabling a subsequent puff of aerosol to be generated.

The first mechanism may be arranged to directly contact the aerosol chamber. Alternatively, the first mechanism may be arranged to contact a component which is attached to the aerosol chamber. For example, the first mechanism may be arranged to contact a pivot arm which is attached to the aerosol chamber and so that the aerosol chamber is pivoted away from contacting the aerosol generating article.

Optionally, the first mechanism further comprises a pivot arm connected to the aerosol chamber.

Optionally, the pivot arm is pivotable about a pivot.

Optionally, the aerosol chamber is arranged to receive aerosol generated from an aerosol generating article.

Optionally, the aerosol provision device further comprises a mouthpiece attached or connected to the aerosol chamber.

Optionally, the aerosol chamber and mouthpiece are detachable from the pivot arm.

Optionally, the first mechanism may further comprise a spigot.

The spigot may be moved between a retracted position and an extended position. In an extended position the spigot may engage with the pivot arm or directly with the aerosol chamber so as to cause the aerosol chamber to pivot out of engagement with the aerosol generating article. When the spigot is moved into a retracted position the pivot arm may be biased by a spring back into a position wherein the aerosol chamber re-engages with the aerosol generating article.

Optionally, the aerosol provision device may further comprise a drive mechanism for rotating or translating the aerosol generating article.

Optionally, the drive mechanism may comprise one or more projections for engaging with one or more corresponding apertures provided in an aerosol generating article.

Optionally, the first mechanism may extend through the one or more projections provided on the drive mechanism.

Optionally, the aerosol chamber may be biased so as to be in either the first position or the second position at rest.

According to another aspect there is provided an aerosol generating system comprising:
an aerosol provision device as described above; and
an aerosol generating article.

Optionally, the aerosol generating article comprises: (i) a substantially circular, oval or polyhedral substrate having one or more portions of aerosol generating material arranged on a first surface of the substrate and/or one or more portions of aerosol generating material arranged on a second surface of the substrate; (ii) a substantially planar substrate having one or more portions of aerosol generating material arranged on a first surface of the substrate and/or one or more portions of aerosol generating material arranged on a second surface of the substrate; or (iii) a prismatic or cylindrical shaped aerosol generating article.

The aerosol generating article may comprise either an open type consumable or a closed type consumable.

According to another aspect there is provided a method of generating aerosol comprising:
inserting an aerosol generating article into an aerosol provision device comprising an aerosol chamber;
causing the aerosol chamber to contact the aerosol generating article;
generating aerosol from the aerosol generating article;
moving the aerosol chamber out of contact with the aerosol generating article;
rotating or translating the aerosol generating article; and then causing the aerosol chamber to re-contact the aerosol generating article.

### BRIEF DESCRIPTION OF THE DRAWINGS

Various embodiments will now be described, by way of example only, and with reference to the accompanying drawings, in which:

FIG. 1 shows an aerosol provision device in combination with an aerosol generating article, wherein the aerosol generating article comprises a plurality of portions of aerosol generating material and wherein the aerosol provision device comprises a single inductive heating element and a movement mechanism for rotating the aerosol generating article relative to the single inductive heating element;

FIG. 2A shows a perspective view of a base portion of an aerosol provision device wherein the aerosol provision device comprises a drive mechanism having a triangular projection for engaging with a corresponding triangular aperture provided in an aerosol generating article and FIG. 2B shows the base portion and a lid portion of an aerosol provision device wherein a disc shaped aerosol generating article is inserted into the aerosol provision device and is engaged with a triangular shaped projection forming part of a drive mechanism for rotating the aerosol generating article;

FIG. 3 shows a cross-sectional view of an aerosol provision device wherein an aerosol chamber is biased into contact with an aerosol generating article; and

FIG. 4 shows a cross-sectional view of an aerosol provision device wherein a spigot is engaged with a pivot arm attached to an aerosol chamber so that the aerosol chamber is disengaged from contacting the aerosol generating article.

### DETAILED DESCRIPTION

Aspects and features of certain examples and embodiments are discussed or described herein. Some aspects and features of certain examples and embodiments may be implemented conventionally and these are not discussed or described in detail in the interests of brevity. It will thus be appreciated that aspects and features of apparatus and methods discussed herein which are not described in detail may be implemented in accordance with conventional techniques for implementing such aspects and features.

According to the present disclosure, a “non-combustible” aerosol provision system is one where a constituent aerosol-generating material of the aerosol provision system (or component thereof) is not combusted or burned in order to facilitate delivery of at least one substance to a user.

In some embodiments, the delivery system is a non-combustible aerosol provision system, such as a powered non-combustible aerosol provision system.

In some embodiments, the non-combustible aerosol provision system is an electronic cigarette, also known as a vaping device or electronic nicotine delivery system (END), although it is noted that the presence of nicotine in the aerosol-generating material is not a requirement.

In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

Typically, the non-combustible aerosol provision system may comprise a non-combustible aerosol provision device and a consumable for use with the non-combustible aerosol provision device.

In some embodiments, the disclosure relates to consumables comprising aerosol-generating material and configured to be used with non-combustible aerosol provision devices. These consumables are sometimes referred to as articles throughout the disclosure.

In some embodiments, the non-combustible aerosol provision system, such as a non-combustible aerosol provision device thereof, may comprise a power source and a controller. The power source may, for example, be an electric power source or an exothermic power source. In some embodiments, the exothermic power source comprises a carbon substrate which may be energised so as to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent.

In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

Aerosol-generating material is a material that is capable of generating aerosol, for example when heated, irradiated or energized in any other way. Aerosol-generating material may, for example, be in the form of a solid, liquid or semi-solid (such as a gel) which may or may not contain an active substance and/or flavorants.

The aerosol-generating material may comprise a binder and an aerosol former.

Optionally, an active and/or filler may also be present. Optionally, a solvent, such as water, is also present and one or more other components of the aerosol-generating material may or may not be soluble in the solvent. In some embodiments, the aerosol-generating material is substantially free from botanical material. In particular, in some embodiments, the aerosol-generating material is substantially tobacco free.

The aerosol-generating material may comprise or be an aerosol-generating film. The aerosol-generating film may be formed by combining a binder, such as a gelling agent, with a solvent, such as water, an aerosol-former and one or more other components, such as active substances, to form a slurry and then heating the slurry to volatilize at least some of the solvent to form the aerosol-generating film. The slurry may be heated to remove at least about 60 wt %, 70 wt %, 80 wt %, 85 wt % or 90 wt % of the solvent. The aerosol-generating film may be a continuous film or a discontinuous film, such an arrangement of discrete portions of film on a support. The aerosol-generating film may be substantially tobacco free.

The aerosol-generating film may comprise or be a sheet, which may optionally be shredded to form a shredded sheet.

The aerosol-generating material may comprise one or more active substances and/or flavors, one or more aerosol-former materials, and optionally one or more other functional material.

An aerosol generator is an apparatus configured to cause aerosol to be generated from the aerosol-generating material. In some embodiments, the aerosol generator is a heater configured to subject the aerosol-generating material to heat energy, so as to release one or more volatiles from the aerosol-generating material to form an aerosol. In some embodiments, the aerosol generator is configured to cause an aerosol to be generated from the aerosol-generating material without heating. For example, the aerosol generator may be configured to subject the aerosol-generating material to one or more of vibration, increased pressure, or electrostatic energy.

A consumable is an article comprising or consisting of aerosol-generating material, part or all of which is intended to be consumed during use by a user. A consumable may comprise one or more other components, such as an aerosol generating material storage area, an aerosol-generating material transfer component, an aerosol generation area, a housing, a wrapper, a mouthpiece, a filter and/or an aerosol-modifying agent. A consumable may also comprise an aerosol generator, such as a heater, that emits heat to cause the aerosol-generating material to generate aerosol in use. The heater may, for example, comprise combustible material, a material heatable by electrical conduction, or a susceptor.

A susceptor is a heating material that is heatable by penetration with a varying magnetic field, such as an alternating magnetic field. The susceptor may be an electrically-conductive material, so that penetration thereof with a varying magnetic field causes induction heating of the heating material. The heating material may be magnetic material, so that penetration thereof with a varying magnetic field causes magnetic hysteresis heating of the heating material. The susceptor may be both electrically-conductive and magnetic, so that the susceptor is heatable by both heating mechanisms. The aerosol provision device that is configured to generate the varying magnetic field is referred to as a magnetic field generator, herein.

Non-combustible aerosol provision systems may comprise a modular assembly including both a reusable aerosol provision device and a replaceable aerosol generating article. In some implementations, the non-combustible aerosol provision device may comprise a power source and a controller (or control circuitry). The power source may, for example, comprise an electric power source, such as a battery or rechargeable battery. In some implementations, the non-combustible aerosol provision device may also comprise an aerosol generating component. However, in other implementations the aerosol generating article may comprise partially, or entirely, the aerosol generating component.

Induction heating is a process in which an electrically-conductive object, referred to as a susceptor, is heated by penetrating the object with a varying magnetic field. The process is described by Faraday's law of induction and Ohm's law. An induction heater may comprise an electromagnet and a device for passing a varying electrical current, such as an alternating current, through the electromagnet. When the electromagnet and the object to be heated are suitably relatively positioned so that the resultant varying magnetic field produced by the electromagnet penetrates the object, one or more eddy currents are generated inside the object. The object has a resistance to the flow of electrical currents and when such eddy currents are generated in the object, their flow against the electrical resistance of the object causes the object to be heated. This process is called Joule, ohmic or resistive heating.

Magnetic hysteresis heating is a process in which an object made of a magnetic material is heated by penetrating the object with a varying magnetic field. A magnetic material can be considered to comprise many atomic-scale magnets, or magnetic dipoles. When a magnetic field penetrates such material, the magnetic dipoles align with the magnetic field. Therefore, when a varying magnetic field, such as an alternating magnetic field, for example as produced by an electromagnet, penetrates the magnetic material, the orientation of the magnetic dipoles changes with the varying applied magnetic field. Such magnetic dipole reorientation causes heat to be generated in the magnetic material.

When an object is both electrically-conductive and magnetic, penetrating the object with a varying magnetic field can cause both Joule heating and magnetic hysteresis heating in the object. Moreover, the use of magnetic material can strengthen the magnetic field, which can intensify the Joule heating.

Various embodiments will now be described.

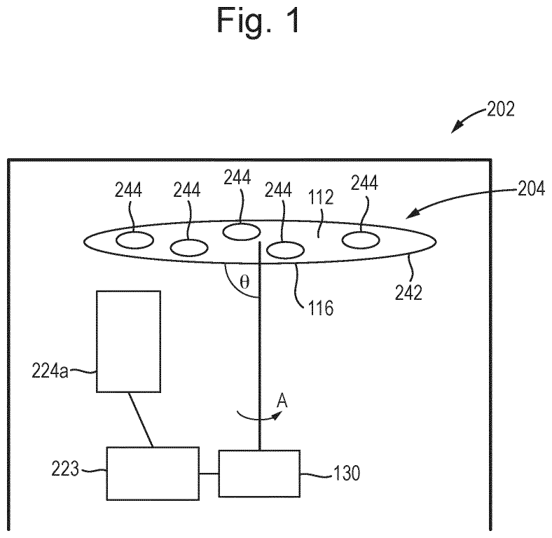

FIG. 1 illustrates a schematic view of a portion of an aerosol provision device 202 according to an arrangement. The aerosol provision device 202 is shown with an aerosol generating article 204 which comprises aerosol generating material located within the aerosol provision device 202. The combination of the aerosol provision device 202 and the aerosol generating article 204 together form an aerosol provision system.

The aerosol generating article 204 has a first (or upper) surface 112 upon which aerosol generating material 244 may be arranged. The aerosol generating article 204 may include a carrier layer 242 (which may be referred to herein as a carrier or a substrate supporting layer) and a susceptor layer on which the aerosol generating material 244 may be disposed. The aerosol generating material 244 may be arranged as a plurality of doses of the aerosol generating material 244. The aerosol generating article 204 may have a second (or lower) surface 116 on the opposite side to the first surface 112. The first surface 112 and/or the second surface 116 may be smooth or rough.

The aerosol provision device 202 may comprise one or more induction heating elements 224a arranged to face the second surface 116 of the aerosol generating article 204. The one or more induction heating elements 224a may be arranged to transfer energy from a power source, such as a battery (not shown), to the aerosol generating material 244 in order to generate aerosol from the aerosol generating material 244.

The aerosol provision device 202 according to an arrangement may have a movement mechanism 130 arranged to move the aerosol generating article 204, and in particular portions (or, in some cases, doses) of aerosol generating material 244. The portions of aerosol generating material 244 may be rotated relative to one or more inductive heating element(s) or induction coil(s) 224a such that portions of the aerosol generating material 244 are presented, in this case individually, to the inductive heating element(s) or induction coil(s) 224a. In the arrangement shown in FIG. 1 the inductive heating element 224a may comprise an induction coil and the aerosol generating article 204 includes a layer that acts as a susceptor.

The aerosol provision device 202 may be arranged such that at least one dose of the aerosol generating material 244 is rotated around an axis A at an angle θ to the second surface 116. Control circuitry 223 may be configured to actuate both the inductive heating element(s) or induction coil(s) 224a and the movement mechanism 130 such that the aerosol generating article 204 rotates so as to align a discrete portion of aerosol generating material 244 in close proximity to the inductive heating element(s) or induction coil(s) 224a. The aerosol generating article 204 may be substantially flat or planar. The carrier layer 242 of the aerosol generating article 204 may be formed of partially or entirely of paper or card.

The aerosol generating article 204 shown in FIG. 1 comprises five doses (or portions) of aerosol generating material 244. In other examples, the aerosol generating article 204 may have more or fewer doses of aerosol generating material 244. In some examples, the aerosol generating article 204 may have the doses of aerosol generating material 244 arranged in discrete doses as shown in FIG. 1.

In other examples, the doses may be in the form of a disc, which may be continuous or discontinuous in the circumferential direction of the aerosol generating article 204. In still other examples, the doses may be in the form of an annulus, a ring or any other shape. The aerosol generating article 204 may or may not have a rotationally symmetrical distribution of doses on the first surface 112 about the axis A. A symmetrical distribution of doses would enable equivalently positioned doses (within the rotationally symmetrical distribution) to receive an equivalent heating profile from the inductive heating element(s) or induction coil(s) 224a upon rotation about the axis A, if desired.

The aerosol generating article 204 of the present example includes aerosol generating material 244 disposed on a susceptor layer of the aerosol generating article 204. However, in other implementations, the aerosol generating article 204 may be formed exclusively of aerosol generating material 244; that is, in some implementations, the aerosol generating article 204 may consist entirely of aerosol generating material 244. In this example, one or more susceptor elements may be provided as part of the aerosol generating device 204.

The aerosol generating article 204 may have a layered structure and may be formed from a plurality of materials. In one example, the aerosol generating article 204 may have a layer formed from at least one of a thermally conductive material, an inductive material, a permeable material or an impermeable material.

In some implementations, the carrier layer 242 or the substrate may be, or may include, a metallic element that is arranged to be heated by a varying magnetic field and hence may act as a susceptor layer. In such implementations, the inductive heating element 224a may include one or more induction coils 224a, which, when energised, cause heating within the metallic element of the aerosol generating article 204. The degree of heating may be affected by the distance between the metallic element or susceptor layer and the induction coil 224a.

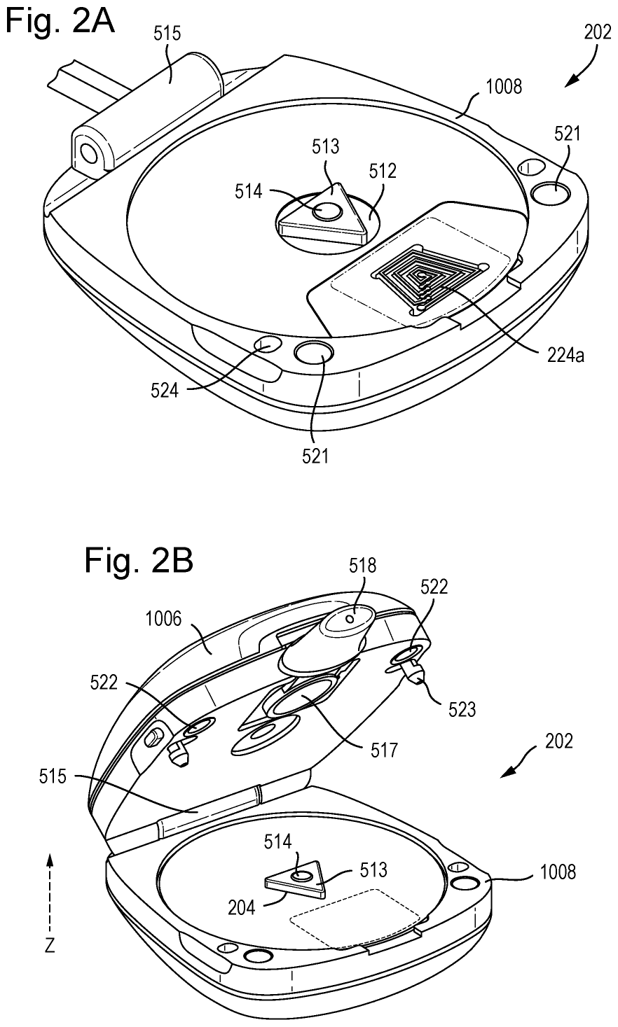

The arrangement shown in FIG. 2 operates by indexing (or moving) the plurality of doses of aerosol generating material 244 relative to the inductive heating element(s) or induction coil(s) 224a. While this arrangement of FIG. 2 may have a slight increase in the complexity of the movement mechanism 130 to provide movement to the aerosol generating article 204, there are benefits to be had by virtue that the aerosol provision device 204 may comprise a single inductive heating element 224a which is used to heat a plurality of portions of aerosol generating material 244. It will be understood that a single heating element 224a requires a single control mechanism (such as control circuitry 223) whereas a plurality of heaters may each require separate control mechanisms. As such, this arrangement can reduce the cost and control complexity in relation to the operation and control of the inductive heating element 224a.

The shape of the aerosol provision device 202 may be cigarette-shape (longer in one dimension than the other two) or may be other shapes. In an example, the aerosol provision device 202 may have a shape that is longer in two dimensions than the other one, for example like a compact-disc player or the like. Alternatively, the shape may be any shape that can suitably house the aerosol generating article 204, one or more inductive heating element(s) or induction coil(s) 224a and the movement mechanism 130.

The aerosol generating article 204 may comprise a carrier component 242 which may be formed of card. The carrier component 242 may form the majority of the aerosol generating article 204 and may act as a base for one or more susceptors or a susceptor layer with aerosol generating material 244 provided or deposited thereupon. The carrier component 242 may be broadly cuboidal or disc shaped in form. The carrier component 242 may have a length (or diameter) of 30-80 mm, a width 7-25 mm and a thickness 0.2 mm. However, it should be appreciated that other arrangements are contemplated wherein the carrier component 242 may have different dimensions as appropriate. In some implementations, the carrier component 242 may comprise one or more protrusions extending in the length and/or width directions of the carrier component 242 to help facilitate handling of the aerosol generating article 204 by the user.

The aerosol generating article 204 may comprise a plurality of discrete portions of aerosol generating material 244 disposed on a surface of the carrier component 242. According to an arrangement the aerosol generating article 204 may comprise two, three, four, five, six, seven, eight, nine, ten, eleven, twelve, thirteen, fourteen, fifteen or more than fifteen discrete portions of aerosol generating material 244.

The discrete portions of aerosol generating material 244 may be disposed in a n×m array. However, it should be appreciated that in other implementations a greater or lesser number of discrete portions may be provided and/or the portions may be disposed in a different format array (e.g. a one by six array). Other arrangements are contemplated wherein the aerosol generating article 204 comprises a disc and separate portions of aerosol generating material 244 are provided in separate segments of the disc.

The aerosol generating material 244 may be disposed at discrete separate locations on a single surface of the component carrier 242. The discrete portions of aerosol generating material 244 are shown as having a circular footprint, although it should be appreciated that the discrete portions of aerosol generating material 244 may take any other footprint, such as square, trapezoidal or rectangular, as appropriate. The discrete portions of aerosol generating material 244 may have a diameter d and a thickness ta. The thickness ta of the discrete portions may take any suitable value, for example the thickness ta may be in the range of 50 μm to 1.5 mm. In some arrangements, the thickness ta may be from about 50 μm to about 200 μm, or about 50 μm to about 100 μm, or about 60 μm to about 90 μm, suitably about 77 μm. In other arrangements, the thickness ta may be greater than 200 μm e.g. from about 50 μm to about 400 μm, or to about 1 mm, or to about 1.5 mm.

The discrete portions of aerosol generating material 244 may be arranged separate from one another such that each of the discrete portions may be energised (e.g. heated) individually or selectively to produce an aerosol.

In some implementations, the portions of aerosol generating material 244 may have a mass no greater than 20 mg, such that the amount of material to be aerosolized by an induction heater or induction coil 224a and associated susceptor element at any one time is relatively low. For example, the mass per portion may be equal to or lower than 20 mg, or equal to or lower than 10 mg, or equal to or lower than 5 mg. It is contemplated that the total mass of the aerosol generating article 204 may be greater than 20 mg.

The aerosol generating article 204 may comprise a plurality of portions of aerosol generating material 244 all formed from the same aerosol generating material. Alternatively, the aerosol generating article 204 may comprise a plurality of portions of aerosol generating material 244 where at least two portions are formed from different aerosol generating materials.

The one or more inductive heating element(s) or induction coil(s) 224a may be positioned such that a surface of the one or more inductive heating element(s) or induction coil(s) 224a forms a part of the surface of a reception region of the aerosol provision device 202. That is, an outer or upper surface of the one or more inductive heating element(s) or induction coil(s) 224a may be flush with an inner surface of the reception region.

The reception region may comprise components which apply a force to the surface of the aerosol generating article 204 in order to press the aerosol generating article 204 onto a surface of the aerosol provision device 202 so as to prevent relative movement of the aerosol generating article 204 when it is desired to generate aerosol from aerosol generating material. The lid portion of the aerosol provision device 202 may be configured to engage with the base portion via a securing mechanism. Accordingly, the lid portion and/or the base portion may comprise components which apply a force to the surface of the aerosol generating article 204 so as to secure the aerosol generating article 204 against relative movement.

Additionally or alternatively, the one or more inductive heater elements or induction coils 224a may be configured to move in a direction towards or away from the aerosol generating article 204, and may be pressed into the surface of carrier component 242 that does not comprise the aerosol generating material 244.

In arrangements wherein the aerosol generating article 204 is configured to move in a specified or desired direction relative to the one or more inductive heater elements or induction coils 224a, the securing mechanism may be configured to engage the lid portion with the base portion so as to hold in position the aerosol generating article 204 to prevent relative movement of the aerosol generating article 204 thereby preventing relative movement in a direction other than in the specified or desired direction.

For example, in arrangements wherein the aerosol generating article 204 is configured to rotate about a rotation axis relative to the one or more inductive heater elements or induction coils 224a so as to present to the one or more inductive heater elements or induction coils 224a a fresh region of aerosol generating material on the aerosol generating article 204, the securing mechanism may be configured to engage the lid portion with the base portion so as to still enable the aerosol generating article 204 to be rotated relative to the one or more inductive heater elements or induction coils 224a whilst preventing relative movement of the aerosol generating article 204 in a direction other than rotation about the rotation axis.

The one or more induction coils 224a may be provided adjacent the reception region 225 and may comprise generally flat coils arranged such that the rotational axis about which a given coil is wound extends into the reception region 225 and is broadly perpendicular to the plane of the carrier component 242 of the aerosol generating article 204.

The control circuitry 223 may comprise a mechanism to generate an alternating current which is passed to any one or more of the induction coils 224a. The alternating current generates an alternating magnetic field which in turn causes the corresponding susceptor(s) or a portion of a susceptor layer to heat up. The heat generated by the susceptor(s) or a portion of a susceptor layer is transferred to the portions of aerosol generating material 244 accordingly.

The control circuitry 223 may be configured to supply current to the induction coils 224a in response to receiving signalling from the touch sensitive panel 229 and/or the inhalation sensor 230.

Various arrangements have been described wherein one or more susceptors are provided as part of aerosol generating article 204. However, other arrangements are contemplated wherein one or more susceptors may be located within or as part of the aerosol provision device 202. For example, one or more susceptors may be provided above the one or more induction coils 224a and may be arranged such that the one or more susceptors contact the lower surface of the carrier component 242 of the aerosol generating article 204.

An aerosol generating article 204 for use with the aerosol provision device 202 may comprise a carrier component 242, one or more susceptor elements and one or more portions of aerosol generating material.

The one or more susceptor elements may be formed from aluminium foil, although it should be appreciated that other metallic and/or electrically conductive materials may be used in other implementations. The carrier component 242 may comprise a number of susceptor elements which correspond in size and location to the discrete portions of aerosol generating material disposed on the surface of the carrier component 242. That is, the susceptor elements may have a similar width and length to the discrete portions of aerosol generating material.

The susceptor elements may be embedded in the carrier component 242. However, in other arrangements, the susceptor elements may be placed or located on the surface of the carrier component 242. According to another arrangement a susceptor may be provided as a single layer substantially covering the carrier component 244. According to an arrangement the aerosol generating article 204 may comprise a substrate or support layer, a single layer of aluminium foil which acts as a susceptor and one or more regions of aerosol generating material 244 deposited upon the aluminium foil susceptor layer.

Although the above has described implementations where discrete, spatially distinct portions of aerosol generating material 244 are deposited on a carrier component 242, it should be appreciated that in other implementations the aerosol generating material 244 may not be provided in discrete, spatially distinct portions but instead be provided as a continuous sheet, film or layer of aerosol generating material 244. In these implementations, certain regions of the sheet of aerosol generating material 244 may be selectively heated to generate aerosol in broadly the same manner as described above. In particular, a region (corresponding to a portion of aerosol generating material) may be defined on the continuous sheet of aerosol generating material 244 based on the dimensions of the one or more inductive heating elements 224a. Each region or portion of aerosol generating material 244 may have a mass no greater than 20 mg, however the total continuous sheet, film or layer may have a mass which is greater than 20 mg.

According to various arrangements the aerosol generating article 204 may comprise a disc shaped or circular consumable.

Although it has been described above that the one or more heating elements 224a are arranged to provide heat to aerosol generating material 244 (or portions thereof) at an operational temperature at which aerosol is generated from the portion of aerosol generating material 244, in some implementations, the one or more inductive heating elements or induction coils 224a and associated susceptor element(s) may be arranged to pre-heat portions of the aerosol generating material to a pre-heat temperature (which is lower than the operational temperature).

At the pre-heat temperature, a lower amount or substantially no aerosol may be generated when the portion is heated at the pre-heat temperature. In particular, in some implementations, the control circuitry 223 may be configured to supply power or energy prior to a first predetermined period starting i.e. prior to receiving signalling signifying a user's intention to inhale aerosol. A lower amount of energy is required to raise the temperature of the aerosol generating material 244 from the pre-heat temperature to the operational temperature, thus increasing the responsiveness of the system. This may be particularly suitable for relatively thick portions of aerosol generating material e.g. having thicknesses greater than 400 μm, which may require relatively larger amounts of energy to be supplied in order to reach an operational temperature.

In addition, when the portions of aerosol generating material are provided on a carrier component 242 the portions may, in some implementations, include weakened regions e.g. through holes or areas of relatively thinner aerosol generating material, in a direction approximately perpendicular to the plane of the carrier component 242. The through holes may provide channels for the generated aerosol to escape and be released to the environment or the air flow through the aerosol provision device 202 rather than causing a potential build-up of aerosol between the carrier component 242 and the aerosol generating material 244. Such build-up of aerosol can reduce the heating efficiency of the system as the build-up of aerosol can, in some implementations, cause a lifting of the aerosol generating material from the carrier component 242 thus decreasing the efficiency of the heat transfer to the aerosol generating material. Each portion of aerosol generating material may be provided with one of more weakened regions as appropriate.

The aerosol provision device 202 may comprise a rotating device configured to rotate, about a rotation axis, the aerosol generating article 204. The rotation device may be configured to rotate the aerosol generating article 204 relative to one or more induction coil(s) 224a so that one or more fresh aerosol generating regions of the aerosol generating article 204 are moved into proximity to the one or more induction coil(s) 224a. A securing mechanism may be configured to enable the aerosol generating article 204 to be rotated relative to the one or more inductions coil(s) 224a whilst preventing relative movement of the aerosol generating article 204 in a direction other than rotation about the rotation axis.

In arrangements, the aerosol generating article 204 may comprise one or more tracks, wherein the lid and/or base portion may be configured to apply a force along the one or more tracks in order to enable the aerosol generating article 204 to be rotated whilst preventing relative movement of the aerosol generating article 204 in a direction other than rotation about the rotation axis. In some arrangements, the one or more tracks may comprise regions of the aerosol generating article 204 which do not include any aerosol generating material. In some arrangements, the one or more tracks may comprise regions of the aerosol generating article 204 comprising metallic foil.

The aerosol provision device 202 may comprise a lid portion which may comprise an aerosol chamber or plenum and a mouthpiece. In some arrangements, the mouthpiece and aerosol chamber or plenum may be integral with the lid portion.

It is contemplated that the aerosol chamber or plenum may be brought into contact with the surface of the aerosol generating article 204 during a session of use. It will be understood that an integrated mouthpiece and lid portion may be provided which may ensure even compression of the aerosol generating article 204. That is, there is substantially no additional mechanical play or variance arising from a connection between the mouthpiece and the lid portion if they are provided as a single integral piece. As a result, the force exerted by the lid portion onto the aerosol generating article 204 may be substantially constant across the upper lid-facing surface of the aerosol generating material.

Other arrangements are contemplated wherein the aerosol provision device 202 may comprise a removable mouthpiece which may be retained within the housing of the aerosol provision device 202 by one or more magnets. It is also contemplated that the aerosol chamber or plenum may also be removable.

FIG. 2A shows a base portion 1008 of an aerosol provision device 202 and shows a rotatable drive mechanism 512 having a triangular shaped projection 513.

The drive mechanism 512 is configured to rotate about a rotation axis an aerosol generating article (not shown in FIG. 2A) which may be loaded, in use, onto the drive mechanism 512. The drive mechanism 512 may be configured to rotate the aerosol generating article relative to one or more inductive heating elements 224a which may be located in the base portion 1008 of the aerosol provision device 202. In particular, the drive mechanism 512 may be arranged to rotate the aerosol generating article so that one or more fresh regions of aerosol generating material provided on the aerosol generating article are moved into proximity of the one or more inductive heating elements 224a.

FIG. 2B shows a perspective view of the aerosol provision device 202 with a disc shaped aerosol generating article 204 loaded onto the base portion 1008. The aerosol provision device 202 comprises a lid portion 1006 which is connected to the base portion 1008 via a hinge 515.

The lid portion 1006 may be secured to the base portion 1008 by the interaction of one or more magnetic elements 521 provided in the base portion 1008 and one or more corresponding magnetic elements 522 provided in the lid portion 1006. According to an arrangement the base portion 1008 may comprise two first magnets 521 configured to be magnetically attracted to two corresponding second magnets 522 provided in the lid portion 1006. The first magnets 521 and the second magnets 522 may have opposite polarities.

The one or more first magnets 521 and/or the one or more second magnets 522 may comprise neodymium iron boron (NdFeB), samarium cobalt (SmCo), alnico, ceramic or ferrite magnets.

Other arrangements are contemplated when one of the permanent magnets 521,522 may be replaced by a magnetisable component or a temporary magnet which may be comprised of iron, iron alloy, nickel, nickel alloy, cobalt, cobalt alloy, gadolinium, gadolinium alloy, dysprosium or dysprosium alloy.

Further arrangements are contemplated wherein the lid portion 1006 and/or the base portion 1008 may comprise one or more electromagnets for securing the lid portion 1006 to the base portion 1008. In such arrangements, the one or more electromagnets may be actuated so as to generate a magnetic field which attracts a corresponding permanent magnet or magnetisable component provided in either the lid portion 1006 or the base portion 1008. Yet further arrangements are contemplated wherein both the lid portion 1006 and the base portion 1008 may comprise an electromagnet. Control circuitry (not shown) may be configured so as to actuate the one or more electromagnets.

One or more projections or lugs 523 may be provided in the lid portion 1006 which may engage with corresponding recesses 524 provided in the base portion 1008 in order to secure the lid portion 1006 to the base portion 1008. Additionally and/or alternatively, one or more projections or lugs may be provided in the base portion 1008 and one or more corresponding recesses may be provided in the lid portion 1006 in order to secure the lid portion 1006 to the base portion 1008.

The lid portion 1006 may further comprise an aerosol chamber (or plenum) 517 which may be attached to a mouthpiece 518. The aerosol chamber 517 may be moved into contact with the surface of the aerosol generating article 204 in use so that aerosol generated from a region of the aerosol generating article 204 in proximity to the one or more inductive heating elements 224a may be captured within the aerosol chamber 517 and then passed or otherwise funnelled onwards to the mouthpiece 518 and hence be inhaled by a user.

It will be understood that the aerosol generating article 204 may be rotated during a session of use and that the aerosol generating article 204 may comprise multiple sectors of aerosol generating material. For example, according to one arrangement the aerosol generating article 204 may comprises twelve sectors which may permit a user to obtain up to twelve puffs per session of use.

After a puff has been taken it may be desired to rotate the aerosol generating article 204 so that a fresh region of aerosol generating material is located in proximity to the one or more inductive heating elements 224a. Accordingly, the aerosol chamber 517 may be disengaged from contacting the surface of the aerosol generating article 204 in order to permit the aerosol generating article 204 to be rotated during a session of use.

According to an arrangement a spigot 514 may be provided in the base portion 1008 of the aerosol provision device 202. The spigot 514 may be located in the center of the drive mechanism 512 and may extend through the center of the triangular shaped projection 513. The spigot 514 is shown in a retracted position in FIGS. 2A and 2B but the spigot 514 may be extended so that the spigot 514 projects beyond the upper surface of the triangular shaped projection 513.

The spigot 514 may be arranged to engage with the aerosol chamber 517 in order to push or displace the aerosol chamber 517 out of engagement with the aerosol generating article 204, thereby permitting the aerosol generating article 204 to be rotated relative to the one or more inductive heating elements 224a. After the aerosol generating article 204 has been rotated, the spigot 514 may be retracted so that the aerosol chamber 517 re-engages with the upper surface of the aerosol generating article 204. A securing mechanism may be provided which may lock the lid portion 1006 to the base portion 1008 during a session of use.

According to various arrangements the aerosol generating article 204 may comprise a substrate layer with a metallic foil layer bonded to the substrate. Aerosol generating material may be provided on the upper surface of the metallic foil layer.

The securing mechanism may be arranged to prevent relative movement of the metallic foil layer with respect to the substrate of the aerosol generating article 204. That is, in use when the aerosol provision device 202 may be configured to heat the aerosol generating article 204 so as to generate aerosol, the securing mechanism may be configured to engage the lid portion 1006 with the base portion 1008 so as to secure the aerosol generating article 204 and prevent the metallic foil layer from delaminating from the substrate during heating.

Although only one inductive heating element or induction coil 224a is shown in FIG. 2A, it will be understood that more than one inductive heating element 224a such as two or three inductive heating elements or induction coils may be provided. In some arrangements, the one or more inductive heating elements or induction coils 224a may comprise one or more induction heating elements comprising one or more induction coils for generating a varying magnetic field so as to heat, in use, one or more susceptor elements of an aerosol generating article 204 (such as a metallic foil) held in position by a securing mechanism. Alternatively, or in addition to, the one or more inductive heating elements or induction coils 224a may be replaced by one or more resistive heating elements.

The one or more inductive heating elements or induction coils 224a may define a planar surface as shown in FIG. 2A. A securing mechanism may be configured to cause the lid portion 1006 to engage with the base portion 1008 so as to hold in position parallel to the planar surface a substantially planar aerosol generating article 204 so as to prevent movement of the substantially planar aerosol generating article 204 in a direction substantially perpendicular to the planar surface (i.e. in the z-direction as shown in FIG. 2B).

The one or more inductive heating elements or induction coils 224a may comprise substantially planar heating elements such as one or more flat spiral induction coils. However, other arrangements are contemplated wherein the one or more inductive heating elements or induction coils 224a may be non-planar but wherein they may still define a planar surface. For example, the one or more inductive heating elements or induction coils 224a may comprise one or more inverted conical induction coils wherein the base of the conical coil(s) defines the planar surface.

The aerosol chamber 517 may be integral with the mouthpiece 518. However, other arrangements are contemplated wherein the aerosol provision device 202 may comprise a removable mouthpiece 518 which may be retained to the aerosol chamber 517 by one or more magnets.

The securing mechanism may comprise a hinge 515 such that the lid portion 1006 is connected to the base portion 1008 via the hinge 515 and may form a clamshell arrangement. That is, the aerosol provision device 202 may be configured to receive an aerosol generating article 204 when the hinge 515 is in an open position e.g. as shown in FIG. 2B. The lid portion 1006 may then be closed and the securing mechanism may be configured to engage the lid portion 1006 with the base portion 1008 so as to hold in position, in use, an aerosol generating article 204 so as to prevent relative movement of the aerosol generating article 204.

The drive mechanism 512 may be configured to rotate the aerosol generating article 204 about a rotation axis. According to other arrangements the drive mechanism 512 may be configured to translate the aerosol generating article 204.

The drive mechanism 512 may be configured to rotate and/or translate the aerosol generating article 204 relative to the one or more heating elements 224a so that one or more fresh aerosol generating regions of the aerosol generating article 204 are moved into proximity to the one or more heating elements 224a.

The drive mechanism 512 may comprise a hub having one or more triangular shaped projections 513 provided on the hub. The one or more triangular shaped projections 513 may be arranged to engage with one or more corresponding apertures or recesses provided in the aerosol generating article 204.

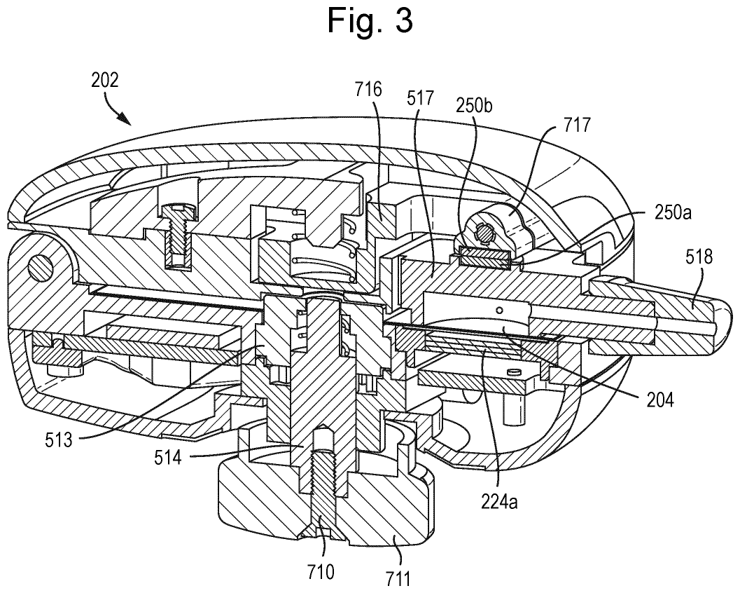

FIG. 3 shows a cross-sectional view showing an aerosol provision device 202 according to an arrangement. The aerosol provision device 202 comprises an aerosol chamber 517 which during a session of use may be biased into contact with an aerosol generating article 204 which has been introduced into the aerosol provision device 202. Aerosol generated from heating a region of the aerosol generating article 204 may be received into the aerosol chamber 517. The aerosol chamber 517 may be both physically and fluidly connected to a mouthpiece 518 so that aerosol received within the aerosol chamber 517 is then onwardly transmitted from the aerosol chamber 517 to the mouthpiece 518. Aerosol transmitted to the mouthpiece 518 may then be inhaled by a user. The mouthpiece 518 may either be integral with the aerosol chamber 517 or alternatively the mouthpiece 518 may be detachable from the aerosol chamber 517.

One or more induction coils or induction heaters 224a may be located in a base portion of the aerosol provision device 202 and may be arranged to heat a region of the aerosol generating article 204 in order to generate aerosol. According to various arrangements the one or more induction coils or induction heaters 224a may have a circular, polygonal or trapezoidal profile and may be arranged to heat a corresponding shaped portion of the aerosol generating article 204.

The aerosol generating article 204 may comprise a base layer or substrate (which may comprise card), a susceptor layer (which may comprise a layer of aluminium foil) and a layer of aerosol generating material which may be provided on the susceptor layer.

According to an arrangement aerosol may be generated from the aerosol generating article 204 on a puff by puff basis. It is contemplated that the aerosol generating article 204 may comprise a relatively thin layer of aerosol generating material which may be provided in the form of a gel. The one or more induction coils or induction heaters 224a may be switched ON and OFF and may be activated when a user desires to take a puff of aerosol. A puff of aerosol may be generated within approx. Is of a user indicating a desire to take a puff of aerosol by activating a user interface.

The aerosol generating article 204 may be disc shaped and may comprise a number of sectors wherein a different sector may be heated for each desired puff. In order to generate aerosol from a fresh region of the aerosol generating article 204 for each puff, the aerosol generating article 204 may be rotated (or translated) in a stepped manner during a session of use in between puffs.

According to various arrangements once a puff of aerosol has been generated then the one or more induction coils or induction heaters 224a may be switched OFF (or the operational set temperature of the one or more induction coils or induction heaters 224a may be lowered to an intermediate temperature). The aerosol chamber 517 may simultaneously or subsequently be disengaged from contact with the aerosol generating article 204 and the aerosol generating article 204 may then be rotated relative to the one or more induction coils or induction heaters 224a. According to an arrangement the aerosol generating article may be rotated <10°, 20-20°, 20-30°, 30-40°, 40-50°, 50-60°, 60-70°, 70-80°, 80-90° or >90° relative to the one or more induction coils or induction heaters 224a each time before or after it is desired to obtain a puff of aerosol.

In order to rotate (or translate) the aerosol generating article 204 multiple times during a session of use the aerosol chamber 517 may need to be disengaged from making physical contact with the aerosol generating article 204 multiple times during the session of use in order for the aerosol generating article 204 to be rotated (or translated) so that a fresh portion of aerosol generating material can be located in proximity to the one or more induction coils or inductive heaters 224a.

In order to disengage the aerosol chamber 517 from making physical contact with the aerosol generating article 204, the aerosol chamber 517 may be pivoted and may be caused to rotate about a pivot so that the aerosol chamber 517 is caused to move in a direction away from the surface of the aerosol generating article 204. According to various arrangements the aerosol chamber 517 may be disengaged from the aerosol generating article 204 so that there is a clearance gap of at least 50 μm, 50-100 μm, 100-500 μm, 500-1000 μm, 1-2 mm, 2-3 mm, 3-4 mm or 4-5 mm between the aerosol chamber 517 and the aerosol generating article 204. As a result of ensuring that the aerosol chamber 517 detaches from making contact with the aerosol generating article 204, the aerosol generating article 204 may then be rotated by the drive mechanism so that a fresh portion of aerosol generating material may be brought into proximity with the one or more induction coils or induction heaters 224a.

The drive mechanism which is used to rotate (or translate) the aerosol generating article 204 may comprise one or more projections 513 which engage with correspondingly shaped aperture(s) provided in the aerosol generating article 204. Accordingly to an arrangement a triangular or other shaped projection 513 may be provided on the drive mechanism and the projection 513 may be arranged to engage with a corresponding triangular or other shaped aperture provided in the aerosol generating article 204.

The projection 513 which extends from the drive mechanism and which is arranged to cause the aerosol generating article 204 to be rotated (or translated) may comprise a central aperture. A spigot 514 may be provided in the base portion of the aerosol provision device 202 and may extend through the central aperture provided in the projection 513.

The spigot 514 may be connected to a cap member 711 via a fastener 710 and may be provided in a bore. The cap member 711 may be moved either towards or away from the base portion of the aerosol provision device 202. According to an arrangement if the cap member 711 is moved towards the base portion of the aerosol provision device 202 then the associated spigot 514 may be caused to move towards the lid portion of the aerosol provision device 202 and engage directly or indirectly with the aerosol chamber 517. If the cap member 711 is moved away from the base portion of the aerosol provision device 202 then the associated spigot 514 may be caused to move away from the lid portion of the aerosol provision device 202 and disengage from the aerosol chamber 517.

According to various arrangements the spigot 514 may be arranged to engage directly with the aerosol chamber 517 or with a component connected to the aerosol chamber 517. For example, according to an arrangement a pivot arm 716 may be provided in the lid portion of the aerosol provision device 202 and the pivot arm 716 may be connected to the aerosol chamber 517 via a connection member 717. The pivot arm 716 may be arranged to pivot about a pivot (not shown). According to an arrangement the spigot 514 may be arranged to engage with the pivot arm 716 so as to cause the pivot arm 716 to rotate about the pivot and to rotate in a direction away from the aerosol generating article 204 so as to lift the aerosol chamber 517 away from contacting the aerosol generating article 204.

According to an arrangement the pivot arm 716 may be biased by a spring so that the aerosol chamber 517 is biased into contact with the aerosol generating article 204.

The spigot 514 may be biased by a spring into a retracted position. The spigot 514 may be provided centrally within the projection 513 provided on the drive mechanism. When the spigot 514 is in a retracted position then a distal end of the spigot 514 may be essentially flush with the surface of the projection 513 as shown in FIG. 3.

The aerosol chamber 517 may comprise a first magnet 250a which is attracted to a second magnet 250b which may be provided on the connection member 717 which is attached to the pivot arm 716. The pivot arm 716 and connected aerosol chamber 517 are shown biased by a spring so that the aerosol chamber 517 contacts the surface of an aerosol generating article 204. A cap member 711 may be pushed inwards to cause the spigot 514 to move from a retracted position into an extended position.

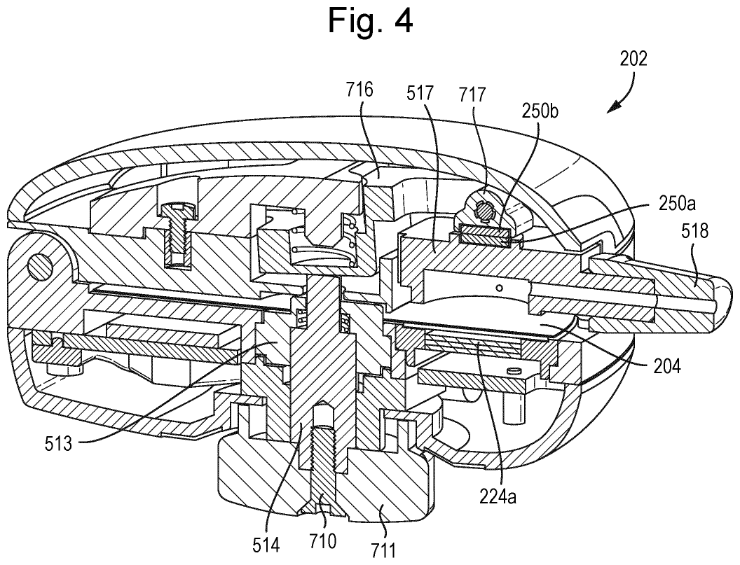

FIG. 4 shows a cross-sectional view showing the aerosol chamber 517 disengaged from the surface of the aerosol generating article 204. According to an arrangement the cap member 711 may be pushed inwards towards the bottom portion of the aerosol provision device 202. The cap member 711 is connected to the spigot 514 via a fastener 710 so that displacement of the cap member 711 towards the base portion of the aerosol provision device 202 will also cause the spigot to be displaced beyond the upper surface of the projection 513 and to move towards the pivot arm 716.

As shown in FIG. 4 the spigot 514 may be arranged to contact the pivot arm 716 and to cause the pivot arm 716 to rotate about a pivot so as to move the aerosol chamber 517 in a direction away from contact with the aerosol generating article 204. As a result, the spigot 514 engages with the pivot arm 716 in order to move the aerosol chamber 517 out of engagement with the aerosol generating article 204.

The pivot arm 716 may be attached to the aerosol chamber 517 via a magnetic connection which may comprise a first magnet 250b provided on a connection member 717 connected to the pivot arm 716 and a second magnet 250a attached to the aerosol chamber 517. The lid portion of the aerosol provision device 204 is shown in a closed position in FIG. 4.

Accordingly, a first mechanism is disclosed comprising a spigot 514 and an optional pivot arm 716 connected to an aerosol chamber 517. The first mechanism may be moved between a first position wherein the aerosol chamber 517 is caused to be in contact with an aerosol generating article 204 and a second position wherein the aerosol chamber 517 is caused no longer to be in contact with the aerosol generating article 204. The aerosol chamber 517 may be biased so as to be in either the first position or the second position at rest.

According to various embodiments an aerosol provision device is provided comprising an aerosol chamber 517 which is moveable between a first position wherein the aerosol chamber 517 is in contact with an aerosol generating article 204 and a second position wherein the aerosol chamber 517 is not in contact with an aerosol generating article 204. A first mechanism may be arranged to move the aerosol chamber 517 between the first and second positions. According to various embodiments the aerosol generating article may comprise a substantially circular or oval substrate having a first surface and a second surface. The substrate may, for example, comprise paper, card or aluminium foil. Other embodiments are contemplated wherein the substrate may comprise multiple layers arranged in a sandwich manner. For example, the substrate may comprise a paper or card substrate having a first aluminium foil layer arranged on a first surface and a second aluminium foil layer arranged on a second surface.

The aerosol generating article may comprise either an open or a closed type of consumable. For example, it will be understood that an open consumable is a type of consumable comprising aerosol generating article wherein the aerosol generating material is provided on one or more outer or outermost surfaces of the aerosol generating article. By contrast, it will be understood that a closed type of consumable comprises an aerosol generating article wherein aerosol generating material is not provided on an outer or an outermost surface of the consumable but rather is provided on one or more internal surfaces. For example, according to various embodiments a closed consumable may be provided wherein one or both outer or outermost surface(s) of the aerosol generating article comprise a gas impermeable layer such as a plastic or other material. For example, embodiments are contemplated wherein an aerosol generating article is provided comprising an innermost substrate having one or more layers of aerosol generating material provided on one or both sides of the substrate and wherein the aerosol generating article is encapsulated or otherwise housed within a housing which is made from a material which is gas impermeable. A closed type of consumable may comprise a housing having an air inlet and an aerosol outlet. The aerosol outlet may comprise a mouthpiece.

According to various embodiments the aerosol generating article may have a length (L), width (W) and thickness (T), wherein the length (L) of the aerosol generating article is greater than the width (W) and/or the thickness (T). The aerosol generating article may have a longitudinal axis and may have a first airflow input end and a second airflow output end. For example, the aerosol generating article may comprise a prism having a first end face and a second end face. The first end face may comprise a region wherein air enters the aerosol generating article in use and the second end face may comprise a region wherein aerosol generated within the aerosol generating article exits the aerosol generating article in use.

Embodiments are contemplated wherein the second end face further comprises a mouthpiece. For example, the aerosol generating article may comprise a distal end (via which air may be arranged to enter the aerosol generating article) and a proximal end (which may comprise a mouthpiece and wherein a user may draw aerosol generated within the aerosol generating article.).

According to various embodiments aerosol generating material may be provided on either a first surface and/or a second surface of a substrate. For example, an aerosol generating article may be provided which is either single or double sided. A single sided aerosol generating article may be activated by a single array of heating elements. A double sided aerosol generating article may be activated by a double array of heating elements which in use are provided on both sides of the aerosol generating article.

Embodiments are contemplated wherein the aerosol generating article may be rotated and/or translated relative to one or more aerosol generators. The one or more aerosol generators may comprise, for example, a single aerosol generator or alternatively a plurality of aerosol generators may be arranged, for example, in an array. Embodiments are contemplated wherein aerosol generators may be provided in a n×m array, wherein n=2, 3, 4, 5, 6, 7, 8, 9, 10 or >10 and wherein m=2, 3, 4, 5, 6, 7, 8, 9, 10 or >10. For example, aerosol generators may be provided in a 2×2 array, a 2×3 array, a 2×4 array, a 2×5 array, a 2×6 array, a 2×7 array, a 2×8 array, a 2×9 array or a 2×10 array.

According to various embodiments the one or more aerosol generators may comprise one or more resistive heaters or resistive heating elements. According to other embodiments the one or more aerosol generators may comprise one or more inductive heaters or inductive heating elements. Embodiments are also contemplated wherein a plurality of resistive and inductive heating elements may be provided.

The aerosol generating article may be arranged to be rotated and/or translated relative to one or more aerosol generators so that the aerosol generating article is located adjacent the one or more aerosol generators and is heated from one side only. Alternatively, the aerosol generating article may be arranged to be rotated and/or translated relative to one or more aerosol generators so that the aerosol generating article is inserted between a first set of aerosol generators and a second set of aerosol generators. According to such an embodiment the aerosol generating article may be arranged to be heated either simultaneously or sequentially from two opposed sides.

Embodiments are also contemplated wherein the aerosol generating article may be prism shaped. For example, the aerosol generating article may comprise a triangular prism, a square shaped prism or a cylindrical prism. For example, the aerosol generating article may comprise a cylindrical aerosol generating article. The aerosol generating article may be rotated and/or translated relative to one or more aerosol generators. For example, the aerosol provision device may comprise a cavity into which a prismatic or cylindrical shaped aerosol generating article may be inserted. A matrix, strip or an array of aerosol generators may be provided at one or more locations around or along the cavity. The aerosol generating article may then be rotated and/or translated relative to the aerosol generators so that different portions of the aerosol generating article may be sequentially or progressively heated or otherwise accessed.

Embodiments are contemplated wherein an aerosol generating article may be translated relative to one of more aerosol generators. For example, the aerosol generating article may comprise a plurality of portions of aerosol generating material and the aerosol generating article may be translated in a longitudinal direction so that a plurality of separate portions of aerosol generating material may be activated or otherwise heated in series or sequentially.

Further embodiments are contemplated wherein the aerosol generating article may comprise a cylinder or more generally a prism. A plurality of aerosol generators may be arranged around or about the cylindrical or prismatic shaped aerosol generating article. It is contemplated that the aerosol generating article may be rotated within a static array of aerosol generators. Alternatively, the aerosol generating article may remain static and a plurality of aerosol generators may be rotated relative to the aerosol generating article. A yet further embodiment is contemplated wherein both the aerosol generating article and one or more aerosol generators are movable. For example, the aerosol generating article may be rotated and/or translated at a first speed v1 and one or more aerosol generators may be rotated and/or translated at a second speed v2. Embodiments are contemplated wherein in a mode of operation v1>v2. Embodiments are contemplated wherein in a mode of operation v1=v2. Embodiments are also contemplated wherein in a mode of operation v1<v2.

According to various embodiments the aerosol generating article may comprise a flat or planar consumable having a longitudinal axis. The aerosol generating article may be translated in a direction parallel to the longitudinal axis. Other embodiments are contemplated wherein the aerosol generating article comprises a cylindrical consumable having a longitudinal axis. The cylindrical consumable may be rotated about the longitudinal and/or may be translated in a direction parallel to the longitudinal axis. The aerosol generating article may be single side or double sided. A double sided consumable may be heated, in use, from both sides.

In order to address various issues and advance the art, this disclosure shows by way of illustration various embodiments. The advantages and features of the disclosure are of a representative sample of embodiments only, and are not exhaustive and/or exclusive. They are presented only to assist in understanding and to teach the claimed invention(s). It is to be understood that advantages, embodiments, examples, functions, features, structures, and/or other aspects of the disclosure are not to be considered limitations on the disclosure as defined by the claims or limitations on equivalents to the claims, and that other embodiments may be utilized and modifications may be made without departing from the scope of the claims. Various embodiments may suitably comprise, consist of, or consist essentially of, various combinations of the disclosed elements, components, features, parts, steps, means, etc. other than those specifically described herein, and it will thus be appreciated that features of the dependent claims may be combined with features of the independent claims in combinations other than those explicitly set out in the claims. The disclosure may include other inventions not presently claimed, but which may be claimed in future.

A detailed listing of all claims that are, or were, in the present application, irrespective of whether the claim(s) remain(s) under examination in the application is presented below. The claims are presented in ascending order and each includes one status identifier. Those claims not cancelled or withdrawn but amended by the current amendment utilize the following notations for amendment: 1. deleted matter is shown by strikethrough for six or more characters and double brackets for five or fewer characters; and 2. added matter is shown by underlining.

1. An aerosol provision device comprising:
an aerosol chamber moveable between a first position wherein the aerosol chamber is in contact with an aerosol generating article and a second position wherein the aerosol chamber is not in contact with an aerosol generating article; and
a first mechanism arranged to move the aerosol chamber between the first and second positions.

2. An aerosol provision device as claimed in claim 1, wherein the first mechanism further comprises a pivot arm connected to the aerosol chamber.

3. An aerosol provision device as claimed in claim 2, wherein the pivot arm is pivotable about a pivot.

4. An aerosol provision device as claimed in claim 1, wherein the aerosol chamber is arranged to receive aerosol generated from an aerosol generating article.

5. An aerosol provision device as claimed in claim 1, further comprising a mouthpiece attached or connected to the aerosol chamber.

6. An aerosol provision device as claimed in claim 5, wherein the aerosol chamber and mouthpiece are detachable from the pivot arm.

7. An aerosol provision device as claimed in claim 1, wherein the first mechanism further comprises a spigot.

8. An aerosol provision device as claimed in claim 1, wherein the aerosol provision device further comprises a drive mechanism for rotating or translating an aerosol generating article.

9. An aerosol provision device as claimed in claim 8, wherein the drive mechanism comprises one or more projections for engaging with one or more corresponding apertures provided in an aerosol generating article.

10. An aerosol provision device as claimed in claim 8, wherein the first mechanism extends through the one or more projections.

11. An aerosol provision device as claimed in claim 1, wherein the aerosol chamber is biased so as to be in either the first position or the second position at rest.

12. An aerosol generating system comprising:
an aerosol provision device as claimed in claim 1; and
an aerosol generating article.

13. An aerosol generating system as claimed in claim 12, wherein the aerosol generating article comprises: (i) a substantially circular, oval or polyhedral substrate having one or more portions of aerosol generating material arranged on a first surface of the substrate and/or one or more portions of aerosol generating material arranged on a second surface of the substrate; (ii) a substantially planar substrate having one or more portions of aerosol generating material arranged on a first surface of the substrate and/or one or more portions of aerosol generating material arranged on a second surface of the substrate; or (iii) a prismatic or cylindrical shaped aerosol generating article.

14. An aerosol generating system as claimed in claim 12, wherein the aerosol generating article comprises either an open type consumable or a closed type consumable.

15. A method of generating aerosol comprising:
inserting an aerosol generating article into an aerosol provision device comprising an aerosol chamber;
causing the aerosol chamber to contact the aerosol generating article;
generating aerosol from the aerosol generating article;
moving the aerosol chamber out of contact with the aerosol generating article;
rotating and/or translating the aerosol generating article; and then causing the aerosol chamber to re-contact the aerosol generating article.
